(19) United States
(12) Patent Application Publication
(10) Pub. No.: US 2024/0245654 A1
(43) Pub. Date: Jul. 25, 2024

(54) ADJUNCTIVE D-CYCLOSERINE AUGMENTATION OF TRANSCRANIAL MAGNETIC STIMULATION (TMS) THERAPY FOR MAJOR DEPRESSIVE DISORDER

(71) Applicant: MCGRX CORP., Calgary (CA)

(72) Inventor: ALEXANDER ROBERT ANGUS MCGIRR, Calgary (CA)

(21) Appl. No.: 18/563,448

(22) PCT Filed: May 26, 2022
(86) PCT No.: PCT/CA2022/050839

§ 371 Date: Nov. 22, 2023

Related U.S. Application Data
(60) Provisional application No. 63/193,643, filed on May 27, 2021.

(51) Int. Cl. A61K 31/42; A61P 25/24

(52) U.S. Cl. CPC A61K 31/42; A61P 25/24

(57) ABSTRACT

There is provided adjunctive D-cycloserine (DCS) augmentation of transcranial magnetic stimulation (TMS) therapy for Major Depressive Disorder (MDD). There is also provided combination therapies and therapeutic methods that include intermittent or continuous theta-burst stimulation (iTBS or cTBS), high- or low-frequency stimulation or combinations thereof with D-cycloserine (DCS) to treat MDD in a patient and/or to improve and/or alleviate and/or reduce frequency of one or more symptoms of MDD as well as complications of MDD, including anxiety, cognitive function, and suicide.

### FIELD OF THE INVENTION

This invention pertains generally to methods of treating depression and, more particularly adjunctive D-cycloserine (DCS) augmentation of transcranial magnetic stimulation (TMS) therapy for Major Depressive Disorder, including intermittent theta-burst stimulation (ITBS), continuous theta-burst stimulation (cTBS) or high- or low-frequency stimulation, or combinations thereof.

### BACKGROUND OF THE INVENTION

Major Depressive Disorder (MDD) is a significant source of global disability characterized by emotional, somatic and cognitive changes and is also associated with increased suicidal ideation and suicide risk. In Canada, the annual prevalence of a current episode of MDD is 3.9% and lifetime prevalence is 9.9%¹. This represents approximately 1.5 million Canadians every year, and over 3.5 million Canadians over the course of their lives. Though the true cost of MDD is not known in Canada, direct healthcare costs exceed $12 billion annually.² The Health Adjusted Life Years burden of MDD in Ontario exceeds that of breast, colorectal, lung and prostate cancers combined,³ and globally this chronic and recurring condition accounts for more Disability Adjusted Life Years than any cancer or infectious disease⁴. MDD is associated with role dysfunction⁵ and lost productivity⁶.

There are effective antidepressant medications and psychological treatments,⁷ however more than 30% of patients are unable to achieve significant benefit even after several interventions and their combinations.⁸ Up to 70% of patients do not achieve remission; relapse typically occurs within months.⁸ Despite an increased uptake in medication and psychological treatments for depression in the last decade, there has not been an associated change in the population prevalence of MDD⁹ and treatment resistant depression has a population prevalence of 2-3%.¹⁰ Alberta Health Services data indicate that each case of treatment resistant depression is associated with 9-fold increase in direct healthcare costs.¹¹ It is therefore critical that better outcomes in MDD treatment and management be achieved.

Non-invasive repetitive Transcranial Magnetic Stimulation (rTMS) of brain regions implicated in MDD has emerged as a treatment option for individuals who have not benefitted from or not tolerated pharmacotherapy. Rates of clinical response with high- and low-frequency rTMS are approximately 50%, and have not improved with newer protocols such as theta-burst stimulation (TBS) delivered intermittently (ITBS) or continuously (cTBS).

Synaptic plasticity is thought to be the mechanism whereby rTMS and TBS result in improvements in depressive symptoms in patients receptive to the positive effects of rTMS or TBS, however this has never been explicitly tested by either blocking or enhancing plasticity mechanisms in conjunction with treatment protocols. This is an important gap, because prior art studying TMS and synaptic plasticity¹² use single interventions and short timescales, typically less than an hour, and target the motor cortex, which is not a brain region utilized as part of treatment protocols. Yet treatment protocols involve daily, or several times daily, stimulations over several weeks targeting regions such as the dorsolateral prefrontal cortex. Accordingly, it is unknown whether rTMS or TBS as a treatment for MDD requires synaptic plasticity, or whether synaptic plasticity can be leveraged to improve outcomes. Prior art is further limited by an overreliance on healthy volunteers¹³⁻¹⁵, whereas a growing literature indicates that individuals with MDD have different responses to TMS plasticity protocols^{17-19, 122}

There exists a need for methods of treating MDD in patients where prior methods of treatment have been unsuccessful or not well tolerated.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide adjunctive D-cycloserine (DCS) augmentation of transcranial magnetic stimulation (TMS) therapy for major depressive disorder. In accordance with an aspect of the invention, there is provided a method of treating Major Depressive Disorder (MDD) in a patient and/or improving and/or alleviating and/or reducing frequency of one or more symptoms of MDD, the method comprising: administering to the patient a low dose of D-cycloserine; and subjecting the patient to transcranial magnetic stimulation, optionally intermittent theta-burst stimulation (iTBS), continuous theta-burst stimulation (cTBS) or high- or low-frequency stimulation, or combinations thereof.

In accordance with another aspect of the invention, there is provided a method of reducing suicidal ideation and suicide risk associated with Major Depressive Disorder (MDD), the method comprising: administering to the patient a low dose of D-cycloserine; and subjecting the patient transcranial magnetic stimulation, optionally intermittent theta-burst stimulation (ITBS), continuous theta-burst stimulation (cTBS) or high- or low-frequency stimulation, or combinations thereof.

In accordance with another aspect of the invention, there is provided a method of improving and/or reversing and/or partially reversing cognitive impairments in MDD, including working memory and overall subjective cognitive function, the method comprising: administering to the patient a low dose of D-cycloserine; and subjecting the patient to transcranial magnetic stimulation, optionally intermittent theta-burst stimulation (ITBS), continuous theta-burst stimulation (cTBS) or high- or low-frequency stimulation, or combinations thereof.

In accordance with another aspect of the invention, there is provided a method of reducing anxiety and anxious distress associated with Major Depressive Disorder (MDD), the method comprising: administering to the patient a low dose of D-cycloserine; and subjecting the patient to transcranial magnetic stimulation, optionally intermittent theta-burst stimulation (ITBS), continuous theta-burst stimulation (cTBS) or high- or low-frequency stimulation, or combinations thereof.

In accordance with another aspect of the invention, there is provided a D-cycloserine for use as a transcranial magnetic stimulation adjuvant, optionally intermittent theta-burst stimulation (ITBS), continuous theta-burst stimulation (cTBS) or high- or low-frequency stimulation, or combinations thereof.

### BRIEF DESCRIPTION OF THE DRAWINGS

These and other features of the invention will become more apparent in the following detailed description in which reference is made to the appended drawings.

FIG. 1 shows time course of motor evoked potential (MEP) peak to peak amplitude from a placebo-controlled randomized controlled study including n=20 healthy participants. Participants received two treatments of iTBS separated by an hour to examine the effect of D-cycloserine on multiple treatments. Repeated spaced iTBS results in facilitation of MEP responses consistent with synaptic plasticity, but this was not impacted by D-cycloserine.

FIG. 2 shows a different metric of synaptic plasticity using a stimulus response curve to examine change after iTBS in a placebo-controlled randomized controlled study including n=20 healthy participants. Adjunctive D-Cycloserine (100 mg) resulted in a significant and sustained facilitation suggestive of enhanced synaptic plasticity. Normalized data is presented in a) and raw data in b).

FIG. 3 shows time course of motor evoked potential (MEP) peak to peak amplitude. Mean motor evoked potential (MEP) amplitude over time in a) healthy (n=12) and Major Depressive Disorder (MDD) participants (n=12) in the placebo condition and in b) MDD participants in placebo and D-cycloserine arms. Error bars represent standard error of the mean.

FIG. 4 shows stimulus response curves at different time points of the experimental paradigm. a) Mean stimulus response curve (SRC) in Major Depressive Disorder (MDD) placebo and D-cycloserine arms at baseline, b) mean SRC 90 minutes following intermittent theta burst stimulation (ITBS), and c) mean SRC the day after iTBS. d) Mean change in SRC from baseline to 16 hours following iTBS in healthy, MDD-placebo, and MDD-D-cycloserine groups. Bonferroni post-hoc healthy-placebo vs MDD-placebo * p<0.05, ** p<0.01. Bonferroni post-hoc MDD-placebo vs MDD-D-cycloserine #p<0.05. Error bars represent standard error of the mean.

FIG. 5 shows relationship between stimulus response curve area under the curve 90 minutes after intermittent theta-burst stimulation (iTBS) and the following day. a) Change in stimulus response curve (SRC) area under the curve (AUC) at 90 minutes post-intermittent theta burst stimulation (iTBS) is negatively correlated to change in SRC AUC the following day in the placebo condition. b) Change in SRC AUC at 90 minutes post-iTBS is positively correlated to change in SRC AUC the following day in the D-cycloserine condition. Bands indicate 95% confidence intervals.

FIG. 6 shows double-blind placebo-controlled randomized control trial (RCT) data in patients with moderate-severe MDD using adjunctive 100 mg DCS (n=25) or placebo (n=25) in conjunction with ITBS. a) ITBS+DCS was associated with a significant improvement in depressive symptoms compared to iTBS+Placebo and higher rates of clinical response. b) Participants randomized to iTBS+DCS have higher rates of clinical response than n=25 participants randomized to iTBS+Placebo. c) Participants randomized to iTBS+DCS have higher rates of clinical remission than n=25 participants randomized to iTBS+Placebo

FIG. 7 shows double-blind placebo-controlled randomized control trial (RCT) data in patients with moderate-severe MDD using adjunctive 100 mg DCS (n=25) or placebo (n=25) in conjunction with ITBS. Participants who were randomized to iTBS+DCS had a) greater improvements in clinical global impression severity scores and b) greater improvements on the clinical global impression scale.

FIG. 8 shows double-blind placebo-controlled randomized control trial (RCT) data in patients with moderate-severe MDD in which n=25 randomized to iTBS+DCS had greater improvements in anxiety than n=25 participants randomized to iTBS+Placebo.

FIG. 9 shows double-blind placebo-controlled randomized control trial (RCT) data in patients with moderate-severe MDD in which n=25 randomized to iTBS+DCS had greater improvements in overall wellbeing as measured by a visual analog scale than n=25 participants randomized to ITBS+Placebo.

FIG. 10 shows THINC-it® data from our RCT in which participants randomized to iTBS+DCS had significant improvements in subjective cognitive function (PDQ) as well as working memory performance on the N-Back task.

FIG. 11 shows evidence for gray matter remodeling at the iTBS target site. Neurite density increases in iTBS+DCS but not iTBS+Placebo randomized participants. The number of participants in clinical and MR data is unequal due to COVID-19 related MR facility closure.

FIG. 12 shows double-blind placebo-controlled randomized control trial (RCT) data in patients with moderate-severe MDD in which n=25 randomized to iTBS+DCS had greater reductions in suicidal ideation than n=25 participants randomized to iTBS+Placebo.

FIG. 13 shows double-blind placebo-controlled randomized control trial (RCT) data in patients with moderate-severe MDD in which n=25 randomized to iTBS+DCS had greater reductions in scores than iTBS+Placebo participants on a computerized neuropsychological test associated with suicide risk called the death-implicit association test.

FIG. 14 shows double-blind placebo-controlled randomized control trial (RCT) data in patients with moderate-severe MDD demonstrating that ingestion of the adjuvant with sufficient time for absorption and distribution is related to treatment response in iTBS+DCS participants only.

### DETAILED DESCRIPTION OF THE INVENTION

Targeted neurostimulation treatments like rTMS and TBS involve delivering trains of stimuli to drive activity dependent changes in the brain, referred to as synaptic plasticity. It is unknown if the efficacy of rTMS and TBS is dependent on plasticity mechanisms, yet there are several lines of evidence indicating impaired TMS-associated plasticity in MDD¹⁶⁻¹⁸, including with the iTBS protocol. Responsiveness to rTMS or TBS in patients with MDD may be improved by improving TMS-associated plasticity in these patients.

One embodiment of the invention, therefore, provides an adjuvant therapy for rTMS or TBS to increase TMS-associated plasticity. Optionally, increases in TMS-associated plasticity includes increased microstructural changes including neurite density and branching.

Accordingly, in one embodiment, combination of rTMS or TBS with adjuvant therapies to improve TMS-associated plasticity is used in methods to treat MDD in a patient and/or to improve and/or alleviate and/or reduce frequency of one or more symptoms of MDD. The one or more symptoms of MDD include feelings of sadness, tearfulness, hopelessness, short temper, irritation, loss of interest/lack of pleasure, memory loss, flat affect, sleep disorders, tiredness, reduced appetite and weight loss and/or feelings of worthlessness.

In one embodiment, combination of rTMS or TBS with adjuvant therapies to improve TMS-associated plasticity is used in methods to improve or restore working memory in a patient with MDD and/or improve or reverse or partially reverse cognitive impairments in a patient with MDD.

In one embodiment, combination of rTMS or TBS with adjuvant therapies to improve TMS-associated plasticity is used in methods to reduce risk of suicide or suicidal ideations in a patient with MDD. In some embodiments, the patient has a history of suicide attempts.

Appropriate high-frequency stimulation protocols are known in the art and include protocols for which there is sham controlled evidence for antidepressant efficacy¹⁹. High-frequency stimulation protocols involve trains of approximately 40 pulses at ≥5 Hz, with inter-train intervals of approximately 10-30 seconds. The intensities can be varied to between about 80% to about 120% of motor threshold. In preferred embodiments, the intensity is between about 100% to 120% of resting motor threshold (rMT).

Appropriate low-frequency stimulation protocols are known in the art and include protocols for which there is sham controlled evidence for antidepressant efficacy¹⁹. Low-frequency stimulation protocols involve trains of pulses approximately 1 Hz, ranging from 300-2400 pulses. The intensities can be varied to between about 80% to about 120% of motor threshold. In preferred embodiments, the intensity is between about 100% to 110% of resting motor threshold (rMT).

Appropriate continuous and intermittent TBS protocols are known in the art and include protocols for which there is sham-controlled evidence for antidepressant efficacy²⁰. The ITBS stimulation frequency is constant with currently available technologies (3-pulse 50-Hz bursts at 5-Hz for 2-seconds trains, with trains every 10 seconds). The cTBS stimulation frequency is constant with currently available technologies (3-pulse 50-Hz bursts at 5-Hz for the duration of the train) The total number of pulses per treatment and the treatment intensity may be varied. The number of pulses per treatment are between about 600 to about 3600. In some embodiments, each treatment includes 600 pulses. In other embodiments, each treatment includes 1200 pulses. In still other embodiments, each treatment includes 1800 pulses. In still other embodiments, each treatment includes 3600 pulses The intensities can be varied to between about 80% to about 120% of motor threshold. In preferred embodiments, the intensity is between about 80% to 90% of resting motor threshold (rMT).

In some embodiments, the iTBS protocol involves 50 Hz bursts, repeated at 5 Hz; 2 seconds on and 8 seconds off; 600 pulses per session delivered at 80% rMT.

In some embodiments, one rTMS or TBS treatment occurs per a day. In other embodiments, multiple rTMS or TBS treatments occur daily, optionally between 2 and 10 treatments daily.

In some embodiments, acute rTMS or TBS treatment courses are delivered over six weeks.

In some embodiments, rTMS or TBS treatment courses are between 1 and 8 weeks of treatment. In some embodiments, rTMS or TBS is continued to be provided after acute treatment courses to prevent relapse.

Synaptic plasticity associated with the rTMS or TBS protocol requires NMDAR signaling,²¹ an ionotropic glutamate receptor that plays a major role in plasticity²². On binding glutamate these tetrameric receptors can initiate membrane depolarization, and through calcium signaling initiate intracellular messenger cascades, gene expression and protein synthesis to alter the strength of synaptic connections²³. Cellular surface expression of NMDAR is itself dynamically regulated and provides a secondary mechanism for long-term modulation of synaptic strength in a manner specific to acute and chronic stress^{24,25}. Accordingly, in one embodiment of the invention, the adjuvant therapy for rTMS or TBS to increase TMS-associated plasticity is an adjuvant therapy that acts through NMDAR signaling.

In one embodiment, a combination of rTMS or TBS with NMDAR agonist or partial agonist is used in methods to treat MDD in a patient and/or to improve and/or alleviate and/or reduce frequency of one or more symptoms of MDD.

In one embodiment, combination of rTMS or TBS with NMDAR agonist or partial agonist is used in methods to improve or restore working memory in a patient with MDD and/or improve or reverse or partially reverse cognitive impairments in a patient with MDD.

In one embodiment, combination of rTMS or TBS with NMDAR agonist or partial agonist is used in methods to reduce risk of suicide or suicidal ideations in a patient with MDD. In some embodiments, the patient has a history of suicide attempts.

One embodiment of the invention provides NMDAR agonist or partial agonist as an adjuvant therapy for rTMS or TBS to increase TMS-associated plasticity.

In embodiments of the invention, using NMDAR agonist or partial agonist as an adjuvant therapy for rTMS or TBS, the NMDAR agonist or partial agonist is provided just before or concurrently with each rTMS or TBS treatment.

DCS is a partial NMDAR agonist that may allow us to harness the molecular machinery of synaptic plasticity to better treat MDD with rTMS or TBS. There are data to indicate that at low dose DCS increases short-term facilitation with high-frequency rTMS¹³, and using transcranial direct current stimulation there is data to suggest plasticity sustained for more than 12 hours than tDCS alone²⁶. DCS preferentially binds to the glycine site of the NR2C NMDAR subunit^{27,28}, which is important because the NR2C subunit is expressed at normal levels in the prefrontal cortex of individuals with MDD²⁹.

In one embodiment, combination of rTMS or TBS with D-cycloserine (DCS) is used in methods to treat MDD in a patient and/or to improve and/or alleviate and/or reduce frequency of one or more symptoms of MDD.

In one embodiment, combination of rTMS or TBS with D-cycloserine (DCS) is used in methods to improve or restore working memory in a patient with MDD and/or improve or reverse or partially reverse cognitive impairments in a patient with MDD.

In one embodiment, combination of rTMS or TBS with D-cycloserine (DCS) is used in methods to reduce risk of suicide or suicidal ideations in a patient with MDD. In some embodiments, the patient has a history of suicide attempts.

One embodiment of the invention, therefore, provides D-cycloserine (DCS) as an adjuvant therapy for rTMS or TBS to increase TMS-associated plasticity.

In embodiments of the invention, DCS is provided as a daily dose just prior to or concurrent with daily rTMS or TBS (2-6 weeks). In some embodiments, DCS is provided about 30 minutes, about 45 minutes, about 60 minutes, about 90 minutes, about 120 minutes or about 180 minutes prior to rTMS or TBS treatment. Optionally, timing of rTMS or TBS treatment is dependent on plasma levels of DCS. Accordingly, in some embodiments, plasma levels of DCS must be above a minimal threshold prior to initiating rTMS or TBS treatment. Optionally, the minimal threshold is about 10 μg/mL.

In some embodiments, the methods of the invention comprise low dose D-cycloserine (DCS). Low dose D-cycloserine (DCS) includes doses ranging from about 1-250 mg of DCS. In preferred embodiments, the dose ranges from about 50 mg to about 150 mg of DCS. Appropriate doses of D-cycloserine may be based on patient's weight or resulting in a 1-30 μg/mL plasma concentration. Optionally, plasma concentration of D-cycloserine is assessed prior to the rTMS or TBS treatment.

In some embodiments, the D-cycloserine is provided as a pharmaceutically acceptable salt of D-cycloserine, a pharmaceutically acceptable ester of D-cycloserine, an alkylated D-cycloserine, or a pharmaceutically acceptable precursor of D-cycloserine.

In some embodiments, the D-cycloserine is provided as a pharmaceutical composition and may include pharmaceutically acceptable carriers or diluents.

The pharmaceutical compositions comprising D-cycloserine can be administered to the patient by any, or a combination, of several routes, such as oral, intravenous, trans-mucosal (e.g., nasal, vaginal, etc.), pulmonary, transdermal, ocular, buccal, sublingual, intraperitoneal, intrathecal or intramuscular. In some embodiments, the pharmaceutical composition is formulated for rapid absorption and distribution.

Oral pharmaceutical formulations, such as tablets, capsule, sprinkle formulations, and oral suspensions, are preferred. Solid compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, lipids, alginic acid, or ingredients for controlled slow release. Disintegrators that can be used include, without limitation, micro-crystalline cellulose, corn starch, sodium starch glycolate and alginic acid. Tablet binders that may be used include, without limitation, acacia, methylcellulose, sodium carboxymethylcellulose, polyvinylpyrrolidone (Povidone), hydroxypropyl methylcellulose, sucrose, starch, and ethylcellulose.

In some embodiments, the D-cycloserine (DCS) is provided as a sublingual formulation.

### EXAMPLES

Example 1: D-Cycloserine increases corticospinal excitability, a measure of synaptic plasticity induced by transcranial magnetic stimulation intermittent theta-burst stimulation. It does so whether intermittent theta-burst stimulation is delivered once or repeatedly in an accelerated design.

Transcranial magnetic stimulation is believed to improve depressive symptoms by changing the function of the brain through a process called synaptic plasticity. The intermittent theta-burst stimulation protocol is known to be dependent on the N-methyl-D-aspartate receptor (NMDA-R). We therefore tested whether low dose D-Cycloserine, a NMDA-R partial agonist, can augment iTBS synaptic plasticity using the motor cortex as a model circuit.

Methods: This experiment was a pre-registered randomized, double-blind, placebo-controlled crossover trial (NCT05081986). Twenty healthy individuals were randomly assigned to one of two experimental arms of the crossover study: a) Placebo-D-cycloserine and b) D-cycloserine-placebo. In the placebo arm, participants received one oral dose of 100 mg microcrystalline cellulose one hour prior to iTBS. In the D-cycloserine 100 mg arm, participants received one oral dose of 100 mg D-cycloserine one hour before ITBS. Participants attended the laboratory on two occasions to complete both arms of the crossover design. Time of arrival was standardized across visits, and visits were separated by at least seven days.

All TMS pulses were delivered using a neuro-navigated MagPro stimulator (X100 system, MagVenture, Denmark) with a biphasic Cool-B70 figure-eight coil. The participant's head and the TMS coil were registered in three-dimensional space using a model MNI brain in the ANT Neuro neuronavigation software (ANT Neuro, Germany). TMS coil placement was controlled using the ANT neuro neuronavigation software; the coil was positioned so that the initial phase of the biphasic pulse delivered an electric field with an orientation posterior to anterior in the brain, relative to the central sulcus. The peak-to-peak amplitude of MEPs were recorded from two surface electromyographic (EMG) electrodes placed on the right first dorsal interosseous muscle (FDI), with the ground electrode placed on the right styloid process. EMG signals were amplified (×1,000), filtered (20-2,000 Hz) using a CED 1401 signal analog/digital converter (Cambridge Electronic Design, UK) and digitized at 5,000 Hz using Signal 6.0 software (Cambridge Electronic Design).

TMS was delivered to the left primary motor cortex to identify the area that, when stimulated, elicited the largest peak-to-peak FDI MEP amplitude (the hotspot). The hotspot area was marked on the neuronavigation software, and all subsequent stimulations were performed at this site. The resting motor threshold (RMT) was determined as the lowest stimulator output, which elicited peak-to-peak MEP amplitudes≥0.05 mV in 5/10 trials.

iTBS was delivered twice, separated by 60 minutes in each arm of the crossover design. ITBS was delivered to the hotspot at 80% RMT. Six-hundred pulses were delivered in stimulation, which consisted of 20 trains of bursts, each composed of 3-pulses at 50 Hz, repeated at 5 Hz with 8-second inter-train intervals.

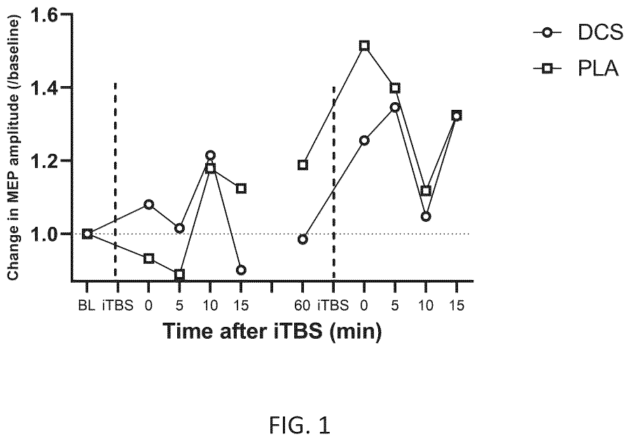

Results: MEP amplitude at baseline was not different between the arms of the crossover design (t=1.4, pₕₒₗₘ=0.571). MEP amplitude prior to the first iTBS (baseline) was not different from MEP amplitude immediately prior to the second iTBS (60 minutes) (both pₕₒₗₘ=1.000, with mean differences below 0.05 mV, FIG. 1).

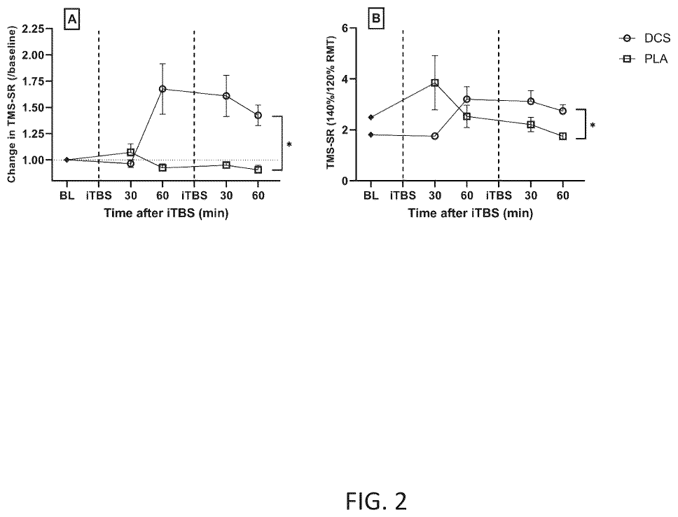

The 140/120% MEP amplitude ratio was used as a measure of the TMS-Stimulus Response Curve. There was a main effect of DRUG on the 140%/120% ratio, where the ratio was higher in the D-cycloserine trial compared to the placebo trial (D-cycloserine=2.4[1.7,3.20], placebo=1.6[0.9,2.3], t(128)=2.6, p=0.010, FIG. 2)

Conclusion: Low-dose D-Cycloserine selectively enhances certain forms of synaptic plasticity after intermittent theta-burst stimulation.

Example 2: Synaptic plasticity impairment in MDD is rescued using intermittent theta-burst trains of TMS stimuli in motor cortex in conjunction with the partial NMDA-R agonist, D-cycloserine, in a randomized double-blind placebo-controlled crossover design.

Methods: A parallel double-blind randomized crossover study using motor cortex transcranial magnetic intermittent theta-burst stimulation among individuals with Major Depressive Disorder (NCT03937596) and healthy individuals (NCT03432689). Eligible participants were randomized with allocation concealment and remained blind to the order in which they received placebo and 100 mg D-cycloserine.

Twelve participants with Major Depressive Disorder and with Hamilton Depression Rating Scale 17-item (HDRS-17) score≥15 (Hamilton, 1960) were recruited. Inclusion criteria were individuals aged 18-60 of any sex, with no changes to their medications in the last 4 weeks. Exclusion criteria were pregnancy, lactation, epilepsy, previous stroke, renal disease, liver disease, current alcohol use disorder (last 3 months), inability to refrain from alcohol use for 24 hours prior to each session, allergy to antibiotics, use of isoniazid, ethionamide, or bupropion, history of psychosis, or intracranial implants.

Data from twelve healthy participants previously recruited via online advertisements and flyers at our centre as part of a separate study (NCT03432689) were used. These healthy participants were aged 18-60 of any sex with no psychiatric diagnoses, and exclusion criteria were identical to those for MDD participants. As needed medications, such as hypnotics, did not constitute an exclusion-criteria for healthy participants. Data from this study suggested that D-cycloserine blunted facilitation of MEPs after iTBS and are described elsewhere 123

Sociodemographic information and current medications were collected at the baseline visit. Participants completed the Quick Inventory of Depressive Symptoms-Self Report (QIDS-SR)⁸ to assess the severity of depressive symptoms, and the Beck Anxiety Inventory (BAI)¹²⁴ and State-Trait Anxiety Inventory (STAI)¹²⁵. Participants also completed the Toronto Side Effects Scale (TSES)¹²⁶ at baseline and the day following iTBS for both study arms.

Clinician administered measures for the MDD sample were the HDRS-17. Clinician administered instruments were not performed in the healthy control sample.

Clinician rated instruments were administered and upon confirmation of eligibility, participants ingested their randomized first capsule (100 mg of D-cycloserine or placebo) before completing their self-report questionnaires. Participants were then taken to the experimental suite where the experiment was explained in situ. They were seated with their hands and elbows rested on a pillow to ensure minimal muscle tone in the upper limbs. The participants' surface anatomical markers were registered to a template magnetic resonance brain image using Neuronavigation software (ANT Neuro, Germany). Then, electromyographic electrodes (Covidien, Ireland) were placed over the right first dorsal interosseous (FDI) muscle with the reference electrode over the radial styloid process. EMG was acquired using the Visor system (ANT Neuro, Germany) at 1 kHz. Between 1-2 hours after ingesting their blinded capsule, a MagPro X100 system with Cool-B70 figure-eight coil (MagVenture, Denmark) was utilized to localize the FDI hotspot. A resting motor threshold (RMT) was then determined, defined as the minimal stimulus intensity needed to generate ≥5/10 MEPs (≥50 microvolts).

Bins of 20 MEPs at 0.25 Hz and 120% RMT were acquired at −15, −10, −5, immediately following iTBS, then +5, +10, +15, +20, +25, +30, +60, and +90 minutes following iTBS. Acquired stimulus response curves (SRC), were also acquired by delivering TMS pulses at 0.25 Hz and randomly varying the stimulus intensity between 100-150% RMT at baseline, 90 minutes following iTBS, and the following day (after re-determining RMT). For each stimulus intensity, a total of four pulses were delivered.

The iTBS protocol consisted of 20 trains of bursts of 3-pulses at 50 Hz, repeated at 5 Hz with an 8 second inter-train interval at 80% RMT. 2.4 Data Processing EMG data were analyzed offline using MATLAB via custom written scripts as previously described¹²³. The peak-to-peak amplitude corrected to the mean value in the 100 ms window preceding the TMS pulse was isolated. EMG traces were individually inspected before inclusion in analyses. For analyses and graphical representation, bins of peak-to-peak values (20 for iTBS time points, 4 for each SRC intensity) were averaged. MEP timecourses were normalized to the mean of baseline values.

Statistical Analyses: Clinical and self-report data were analyzed using SPSS v26 (IBM, Chicago, IL), with paired or independent sample Student t-tests for continuous data and Chi-Square test for dichotomous data. EMG data were analyzed with repeated measures two-way ANOVA and Bonferonni post-hoc tests. Outliers were identified using Tukey's Fences. Alpha was defined as ≤0.05.

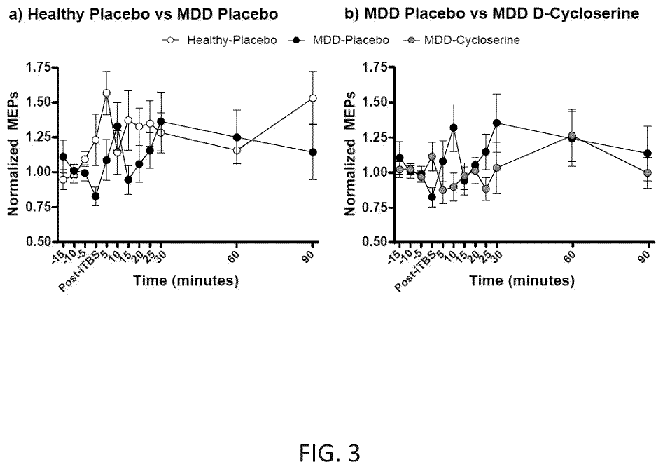

Results: Depressed participants had decreased MEP facilitation following iTBS compared to healthy participants (FIG. 3a; Time F(11,220)=2.15, p=0.018; Group F(1,220)=1.38, p=0.254; Time*Group F(11,220)=2.10, p=0.021), however no timepoint differences survived Bonferroni post-hoc testing.

As with the healthy individuals in Example 1, D-Cycloserine did not impact MEP time course following iTBS and there was no difference between placebo and D-cycloserine treated arms (FIG. 3b; Time F(11,198)=1.40, p=0.175; Treatment F(1,198)=0.982, p=0.335; Time*Treatment F(11,198)=1.51, p=0.130).

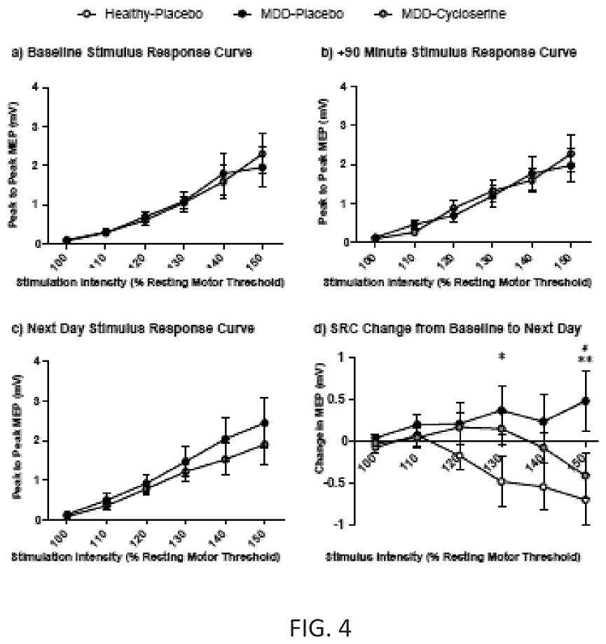

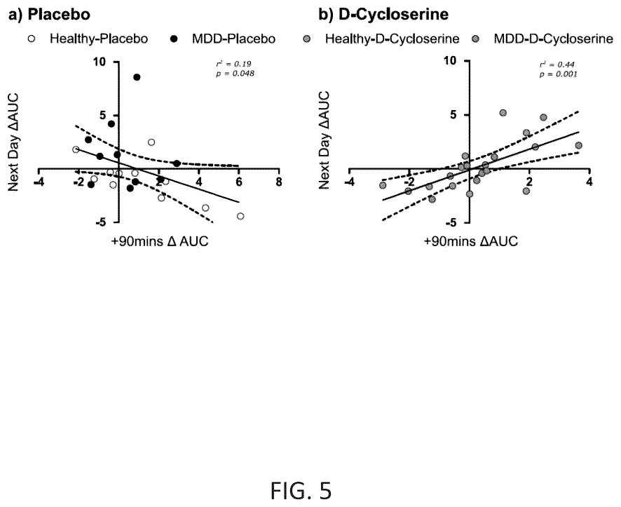

Shifts within an individual's SRC provide a measure of activity dependent change, and therefore SRCs for placebo and D-cycloserine arms was examined at baseline, 90 minutes after iTBS and the day following iTBS (FIG. 4a-c). For normative comparison, data from healthy participants that had ingested a placebo capsule prior to this same design was included (FIG. 4d). Change in SRC the following day revealed opposite patterns of long-term adaptation in healthy and depressed participants, and that D-cycloserine treatment normalized this process in MDD (FIG. 4d; Intensity F(5,145)=1.388, p=0.232; Group F(2,145)=3.364, p=0.049; Intensity*Group F(10,145)=1.898, p=0.0499). The association between changes in the area under the curve (AUC) of the SRC 90 minutes after stimulation (ΔAUC 90 min) and the following day (ΔAUC NextDay) were examined. A significant drug by ΔAUC 90 min effect (Treatment F(2,43)=0.58, p=0.56; ΔAUC_90 min F(1,43)=0.00, p=0.95; Treatment* ΔAUC 90 min F(1,43)=8.16, p=0.007) was found. In the placebo healthy and MDD groups, a weak negative relationship between ΔAUC 90 min and ΔAUC NextDay was identified (FIG. 5a; F(1,19)=4.45, r2=0.19, p=0.048), whereas a positive relationship was seen in the D-cycloserine groups (FIG. 5b; F(1,19)=14.92, r2=0.44, p=0.001).

Conclusion: The NMDA-R partial-agonist D-cycloserine normalizes and stabilizes synaptic plasticity in MDD after iTBS and leads to persistent changes following iTBS.

Example 3: DCS as an adjuvant to iTBS enhances treatment effects and improves depression outcomes in MDD.

Background: Transcranial magnetic stimulation protocols for treatment resistant major depressive disorder (MDD) are believed to depend on synaptic plasticity. The theta-burst stimulation (TBS) protocol is known to be N-methyl-D-aspartate (NMDA) receptor dependent, yet it is unknown whether enhancing NMDA receptor signaling impacts treatment outcomes. Here, we test whether low doses of the NMDA receptor partial-agonist, D-Cycloserine, can enhance intermittent TBS (ITBS) treatment outcomes in MDD.

Methods: In this preregistered (NCT03937596) single site 4-week randomized double-blind placebo-controlled trial, fifty participants with treatment resistant MDD were randomized 1:1 to ITBS+Placebo or iTBS+D-Cycloserine (100 mg). Participants were asked to take the adjunct or placebo at least 60 minutes prior to daily iTBS treatments for the first two weeks, and iTBS continued without an adjunct for weeks three and four. We utilized a MagPro X100 stimulator (MagVenture, Denmark) and a COOL-B70 coil. Targeting of the left DLPFC involved surface anatomy and the Beam F3 method³⁰. Although anatomical and functional neuroimaging was collected as part of the study protocol, targeting utilizing surface anatomy was chosen to increase generalizability to clinical settings where neuroimaging derived targets are not feasible. This target was registered to either the participant's anatomical MRI or to a template brain for those who could not complete neuroimaging (Visor2, ANT Neuro, the Netherlands). Resting motor threshold (rMT) was determined using electromyographic (EMG) electrodes placed over the first dorsal interosseous muscle, with threshold determined as the stimulus intensity required to elicit a minimum of 5 out of 10 EMG responses of >50 uV.

Patients all received active-iTBS, consisting of a total of 600 pulses per session delivered in twenty trains of triplets at 50 Hz repeated at 5 Hz (2 s on 8 s off) at 80% rMT. Participants received daily treatments Monday through Friday for a total of 20 treatments.

The primary outcome was change in depressive symptoms as measured by the Montgomery Asberg Depression Rating Scale (MADRS) at the conclusion of treatment. Secondary outcomes included clinical response (≥50% improvement in MADRS), clinical remission (MADRS≤10), and clinical global impression (CGI).

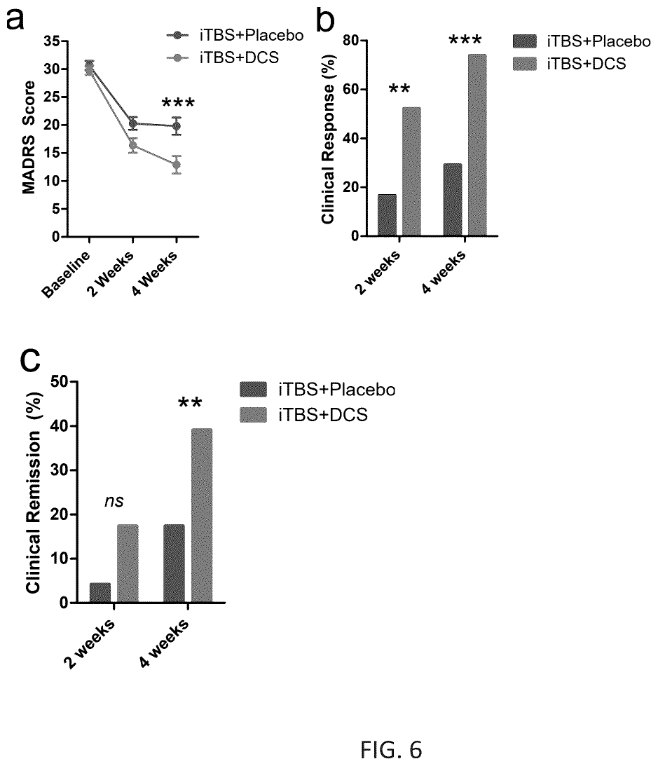

Results: The primary analysis rejected the null hypothesis of equal change in the two groups between baseline and 4 weeks (t(39)=−2.47, p=0.0178). mITT analyses examining change in MADRS scores demonstrated greater reductions in depressive symptoms in the iTBS+DCS group compared to the iTBS+Placebo group (FIG. 6a; Mixed Model Likelihood Ratio (MMLR) Chi-Square=9.97, p=0.0068). This corresponds to a mean difference of 6.15 (95% CI: 2.43-9.88, p=0.001) on the MADRS and a Hedge's g of 0.99 (95% CI: 0.34-1.62).

Per treatment analyses revealed that iTBS+Placebo and iTBS+DCS groups had significantly different MADRS scores at both 2 weeks (mean difference 3.97, 95% CI: 0.43-7.51, p=0.014) and 4 weeks (mean difference 7.09, 95% CI: 2.66-11.52, p=0.0012).

ITBS+DCS was associated with a higher rate of clinical response than ITBS+Placebo at both 2 weeks (ITBS+placebo 16.7% vs iTBS+DCS 52.2%; OR 5.45, 95% CI: 1.41-21.03, p=0.014) and 4 weeks (iTBS+placebo 29.2% vs iTBS+DCS 73.9%; OR 6.88, 95% CI: 1.91-24.77, p=0.003; FIG. 6b). Rates of clinical remission did not separate at 2 weeks (ITBS+placebo 4.2% vs ITBS+DCS 17.4%) but were statistically significantly different by 4 weeks (ITBS+placebo 4.2% vs iTBS+DCS 39.1%; OR 14.78, 95% CI: 1.68-129.52, p=0.014; FIG. 6c).

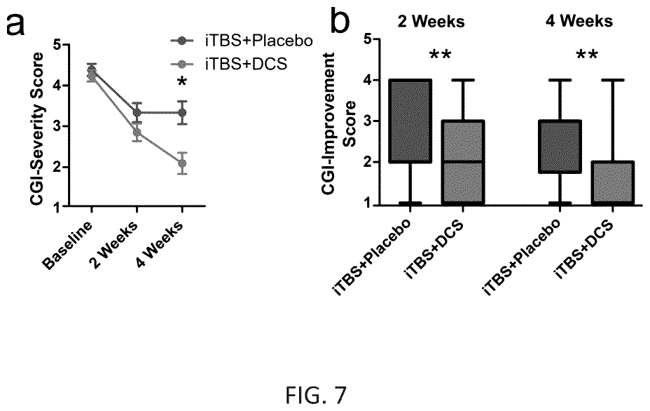

The CGI-severity scale demonstrated greater improvements in the ITBS+DCS compared to the ITBS+Placebo group (MMLR Chi-Square=13.13, p=0.0014, FIG. 7a). When analyzing CGI-Improvement scores, these were not normally distributed and therefore we utilized a Mann Whitney test. This revealed greater improvement in the iTBS+DCS group relative to the ITBS+Placebo group at 2 weeks (z=2.38, p=0.017) and 4 weeks (z=3.24, p=0.0012; FIG. 7b).

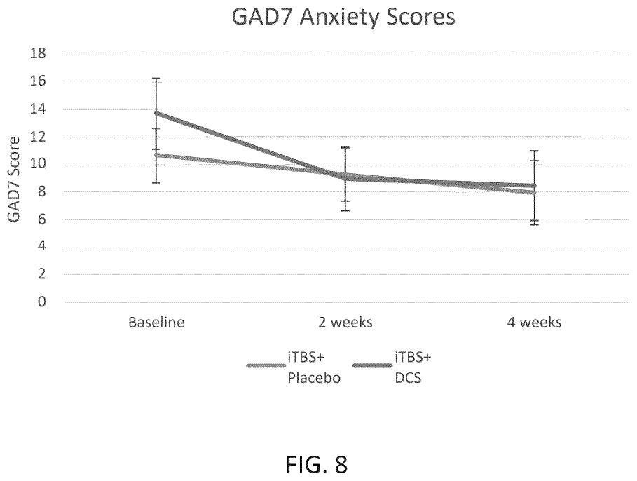

Self-reported anxiety symptoms using the GAD-7 revealed a greater improvement in the ITBS+DCS group compared to the iTBS+Placebo group (MMLR Chi-Square=6.67, p=0.035; FIG. 8).

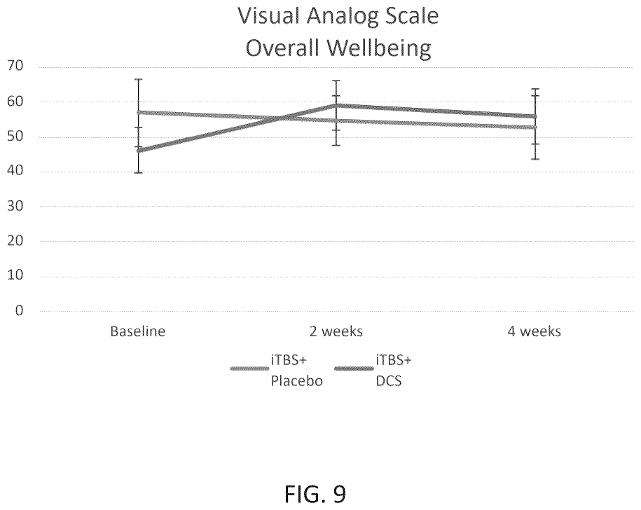

A visual analog scale for overall wellbeing showed a greater improvement in the iTBS+DCS group compared to the iTBD+Placebo group (MMLR Chi-Square=7.94, p=0.018, FIG. 9).

Conclusions: Adjunctive D-Cycloserine improves transcranial magnetic stimulation treatment outcomes in MDD using iTBS.

Example 4: DCS in combination with iTBS reduces objective and subjective cognitive dysfunction.

The efficacy of 100 mg DCS as an adjunctive strategy to iTBS to reduce MDD-related cognitive impairment and/or improve cognition in MDD was be tested in the randomized controlled trial described in the above Example.

Participants completed the Cognitive Failures Questionnaire (CFQ31) and the Perceived Deficits Questionnaire (PDQ) as well as the THINC-it computerized neurocognitive battery at baseline and after treatment.

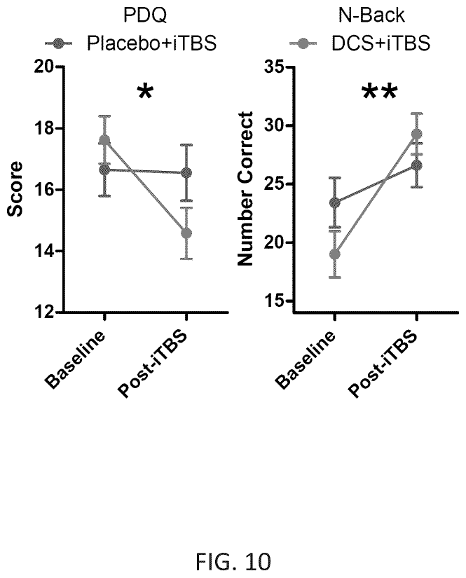

iTBS+DCS participants had significantly greater improvements in their subjective cognitive function as measured by the Perceived Deficits Questionnaire and objective cognitive function on the N-Back task of working memory (FIG. 10).

Example 5: DCS in combination with iTBS induces leads to microstructural remodeling of the TMS target site.

Neurite Density and Dispersion Orientation Index (NODDI)³² is an MRI tool that provides a proxy measure of neurite morphology and arborization that has been validated against histology in model species^{33,34}. While more commonly applied to white matter, NODDI has discriminating power in cortical gray matter in both health and disease³⁵⁻³⁷. Neurite density metrics reflect the volume within neurites, whereas orientation dispersion metrics capture the tortuosity or complexity of neural processes, both of which are affected in MDD.

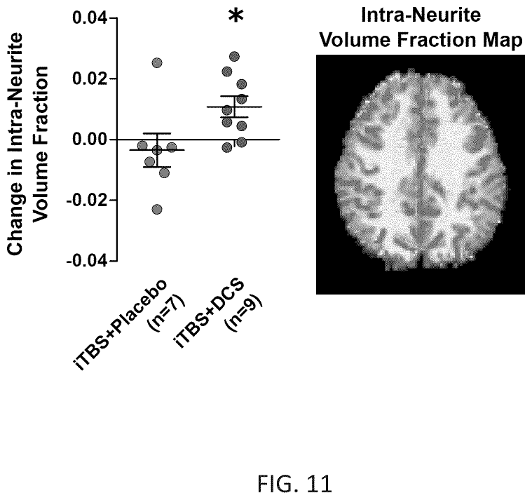

In the randomized placebo-controlled trial discussed in the Example above, diffusion magnetic resonance data to quantify Neurite Density and Dispersion Orientation Index (NODDI)³² were acquired using a 3T General Electric Discovery MR750 scanner at the Seaman Family MR Research Center. This method provides a proxy measure of histopathological features in brain white and gray matter to describe neurite morphology and arborization. Despite small sample, a large effect was observed for increased neurite density at the target site in iTBS+DCS treated participants (FIG. 11).

Example 6: DCS in combination with ITBS reduces suicidal ideation and suicide risk as measured by the death-implicit association test.

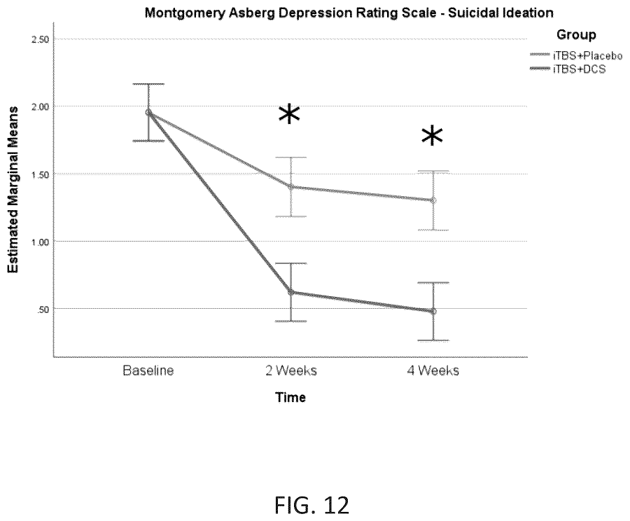

In the randomized placebo-controlled trial discussed in the Example above, participants receiving iTBS+DCS had a rapid resolution of suicidal ideation that was statistically greater than those receiving iTBS+Placebo (FIG. 12).

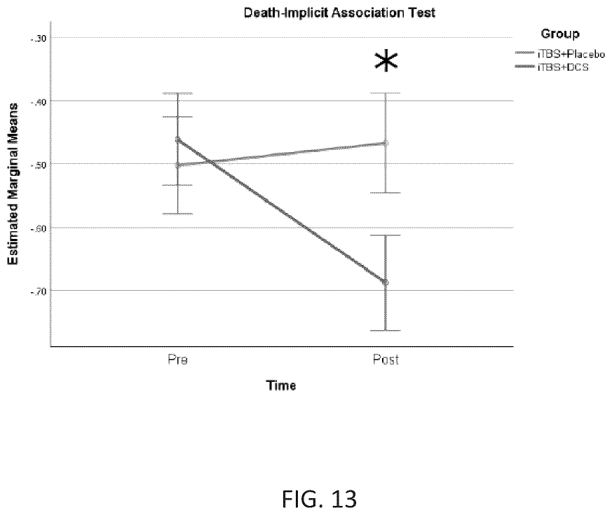

Participants also completed a computerized neuropsychological test called the death-implicit association test that is associated with suicide attempts, both retrospectively and prospectively. Performance on this test showed improvement in the iTBS+DCS group but not the iTBS+Placebo group, and this effect persisted when statistically controlling for differences in depressive symptom improvement (FIG. 13).

Example 7: Augmentation of TMS depends on DCS having sufficient time for absorption and distribution to be present in the brain during stimulation.

In the randomized placebo-controlled trial discussed in the Example above, participants were instructed to ingest their capsules at least 60 minutes prior to TMS treatment. This was to allow sufficient absorption and distribution of oral DCS³⁸.

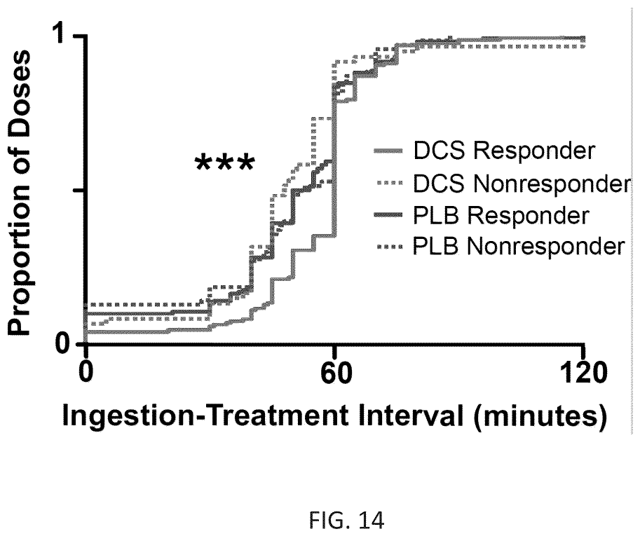

We examined whether there was a relationship between adherence to the protocol, and therefore presence of DCS in the brain during treatment, and treatment outcome by comparing clinical responders and non-responders in the two intervention groups. This resulted in a 4-group comparison. A Kruskal-Wallis test examined the time between ingestion and treatment, and found a significant effect of group (H(3)=25.15, p<0.001, FIG. 14). Bonferroni post-hoc comparison revealed that iTBS+DCS responders had longer intervals between ingestion and treatment than iTBS+DCS non-responders (p<0.001) iTBS+Placebo responders (p=0.044) and ITBS+Placebo nonresponders (p<0.001).

This demonstrates that treatment outcome was related to sufficient time for absorption and distribution of D-Cycloserine in the brain when TMS was delivered, and that adherence to the protocol had no relation to outcome in placebo treated participants.

### REFERENCES

1. Patten S B, Williams J V, Lavorato D H, Wang J L, McDonald K, Bulloch A G. Descriptive epidemiology of major depressive disorder in Canada in 2012. Can J Psychiatry. January 2015; 60(1):23-30. doi:cjp-2015-vol60-january-23-30 [pii] 10.1177/070674371506000106 [doi]
2. Tanner J A, Hensel J, Davies P E, Brown L C, Dechairo B M, Mulsant B H. Economic Burden of Depression and Associated Resource Use in Manitoba, Canada. Can J Psychiatry. May 2020; 65(5):338-346. doi: 10.1177_0706743719895342 [pii] 10.1177/0706743719895342 [doi]
3. Ratnasingham S, Cairney J, Manson H, Rehm J, Lin E, Kurdyak P. The burden of mental illness and addiction in ontario. Can J Psychiatry. September 2013; 58(9):529-37. doi: 10.1177/070674371305800908 [doi]
4. Global, regional, and national disability-adjusted life-years (DALYs) for 359 diseases and injuries and healthy life expectancy (HALE) for 195 countries and territories, 1990-2017: a systematic analysis for the Global Burden of Disease Study 2017. Lancet. Nov. 10 2018; 392(10159): 1859-1922. doi:S0140-6736(18)32335-3 [pii] 10.1016/S0140-6736(18)32335-3 [doi]
5. Druss B G, Hwang I, Petukhova M, Sampson N A, Wang P S, Kessler R C. Impairment in role functioning in mental and chronic medical disorders in the United States: results from the National Comorbidity Survey Replication. Mol Psychiatry. July 2009; 14(7): 728-37. doi:mp200813 [pii] 10.1038/mp.2008.13 [doi]
6. Alonso J, Petukhova M, Vilagut G, et al. Days out of role due to common physical and mental conditions: results from the WHO World Mental Health surveys. Mol Psychiatry. December 2011; 16(12): 1234-46. doi:mp2010101 [pii] 10.1038/mp.2010.101 [doi]
7. Lam R W, Kennedy S H, Parikh S V, et al. Canadian Network for Mood and Anxiety Treatments (CANMAT) 2016 Clinical Guidelines for the Management of Adults with Major Depressive Disorder: Introduction and Methods. Can J Psychiatry. September 2016; 61(9):506-9. doi: 10.1177/0706743716659061
8. Rush A J, Trivedi M H, Wisniewski S R, et al. Acute and longer-term outcomes in depressed outpatients requiring one or several treatment steps: a STAR*D report. Am J Psychiatry. November 2006; 163(11): 1905-17. doi:163/11/1905 [pii] 10.1176/ajp.2006.163.11.1905 [doi]
9. Patten S B, Williams J V, Lavorato D H, Wang J L, McDonald K, Bulloch A G. Major Depression in Canada: What Has Changed over the Past 10 Years? Can J Psychiatry. February 2016; 61(2):80-5. doi:61/2/80 [pii] 10.1177_0706743715625940 [pii] 10.1177/0706743715625940 [doi]
10. Nemeroff C B. Prevalence and management of treatment-resistant depression. J Clin Psychiatry. 2007; 68 Suppl 8:17-25.
11. Treatment Resistant Depression: The Alberta Perspective (Alberta Health Services) (2019).
12. Ziemann U, Reis J, Schwenkreis P, et al. TMS and drugs revisited 2014. Journal Article Review. Clin Neurophysiol. October 2015; 126(10): 1847-68.
13. Brown J C, DeVries W H, Korte J E, et al. NMDA receptor partial agonist, d-cycloserine, enhances 10 Hz rTMS-induced motor plasticity, suggesting long-term potentiation (LTP) as underlying mechanism. Brain Stimul. May-June 2020; 13(3):530-532. doi:S1935-861X(20)30005-X [pii] 10.1016/j.brs.2020.01.005 [doi]
14. Selby B, MacMaster F P, Kirton A, McGirr A. d-cycloserine blunts motor cortex facilitation after intermittent theta burst transcranial magnetic stimulation: A double-blind randomized placebo-controlled crossover study. Brain Stimul. July-August 2019; 12(4):1063-1065. doi:S1935-861X(19)30105-6 [pii] 10.1016/j.brs.2019.03.026 [doi]
15. Teo J T, Swayne O B, Rothwell J C. Further evidence for NMDA-dependence of the after-effects of human theta burst stimulation. Letter Randomized Controlled Trial. Clin Neurophysiol. July 2007; 118(7):1649-51.
16. Kuhn M, Mainberger F, Feige B, et al. State-Dependent Partial Occlusion of Cortical LTP-Like Plasticity in Major Depression. Neuropsychopharmacology. May 2016; 41(6): 1521-9. doi:npp2015310 [pii] 10.1038/npp.2015.310 [doi]
17. Player M J, Taylor J L, Weickert C S, et al. Neuroplasticity in depressed individuals compared with healthy controls. Neuropsychopharmacology. October 2013; 38(11): 2101-8. doi:npp2013126 [pii] 10.1038/npp.2013.126 [doi]
18. Vignaud P, Damasceno C, Poulet E, Brunelin J. Impaired Modulation of Corticospinal Excitability in Drug-Free Patients With Major Depressive Disorder: A Theta-Burst Stimulation Study. Front Hum Neurosci. 2019; 13:72. doi:10.3389/fnhum.2019.00072 [doi]
19. Brunoni A R, Chaimani A, Moffa A H, et al. Repetitive Transcranial Magnetic Stimulation for the Acute Treatment of Major Depressive Episodes: A Systematic Review With Network Meta-analysis. JAMA Psychiatry. Feb. 1 2017; 74(2): 143-152. doi:2594387 [pii] 10.1001/jamapsychiatry.2016.3644 [doi]
20. Li C T, Chen M H, Juan C H, et al. Efficacy of prefrontal theta-burst stimulation in refractory depression: a randomized sham-controlled study. Journal Article Randomized Controlled Trial Research Support, Non-U.S. Gov't. Brain. July 2014; 137(Pt 7): 2088-98.
21. Huang Y Z, Chen R S, Rothwell J C, Wen H Y. The after-effect of human theta burst stimulation is NMDA receptor dependent. Journal Article Randomized Controlled Trial Research Support, Non-U.S. Gov't. Clin Neurophysiol. May 2007; 118(5):1028-32.
22. Traynelis S F, Wollmuth L P, McBain C J, et al. Glutamate receptor ion channels: structure, regulation, and function. Pharmacol Rev. September 2010; 62(3):405-96. doi:10.1124/pr.109.002451
23. Popoli M, Yan Z, McEwen B S, Sanacora G. The stressed synapse: the impact of stress and glucocorticoids on glutamate transmission. Nat Rev Neurosci. Nov. 30 2011; 13(1):22-37. doi:nrn3138 [pii] 10.1038/nrn3138 [doi]
24. Yuen E Y, Liu W, Karatsoreos I N, Feng J, McEwen B S, Yan Z. Acute stress enhances glutamatergic transmission in prefrontal cortex and facilitates working memory. Proc Natl Acad Sci USA. Aug. 18 2009; 106(33):14075-9. doi:0906791106 [pii] 8967 [pii] 10.1073/pnas.0906791106 [doi]
25. Yuen E Y, Wei J, Liu W, Zhong P, Li X, Yan Z. Repeated stress causes cognitive impairment by suppressing glutamate receptor expression and function in prefrontal cortex. Neuron. Mar. 8 2012; 73(5):962-77. doi:S0896-6273(12)00083-9 [pii] 10.1016/j.neuron.2011.12.033 [doi]
26. Nitsche M A, Jaussi W, Liebetanz D, Lang N, Tergau F, Paulus W. Consolidation of human motor cortical neuroplasticity by D-cycloserine. Neuropsychopharmacology. August 2004; 29(8): 1573-8. doi:1300517 [pii] 10.1038/sj.npp.1300517 [doi]
27. Dravid S M, Burger P B, Prakash A, et al. Structural determinants of D-cycloserine efficacy at the NR1/NR2C NMDA receptors. J Neurosci. Feb. 17 2010; 30(7):2741-54. doi:30/7/2741 [pii] 3573526 [pii] 10.1523/JNEUROSCI.5390-09.2010 [doi]
28. Sheinin A, Shavit S, Benveniste M. Subunit specificity and mechanism of action of NMDA partial agonist D-cycloserine. Neuropharmacology. August 2001; 41(2):151-8. doi: S0028390801000739 [pii] 10.1016/s0028-3908(01)00073-9 [doi]
29. Beneyto M, Meador-Woodruff J H. Lamina-specific abnormalities of NMDA receptor-associated postsynaptic protein transcripts in the prefrontal cortex in schizophrenia and bipolar disorder. Neuropsychopharmacology. August 2008; 33(9):2175-86. doi: 1301604 [pii] 10.1038/sj.npp.1301604 [doi]
30. Beam W, Borchardt J J, Reeves S T, George M S. An efficient and accurate new method for locating the F3 position for prefrontal TMS applications. Brain Stimul. January 2009; 2(1):50-4. doi:S1935-861X(08)00337-9 [pii] 10.1016/j.brs.2008.09.006 [doi]
31. Broadbent D E, Cooper P F, FitzGerald P, Parkes K R. The Cognitive Failures Questionnaire (CFQ) and its correlates. Br J Clin Psychol. February 1982; 21(1): 1-16. doi: 10.1111/j.2044-8260.1982.tb01421.x [doi]
32. Zhang H, Schneider T, Wheeler-Kingshott C A, Alexander D C. NODDI: practical in vivo neurite orientation dispersion and density imaging of the human brain. Neuroimage. Jul. 16 2012; 61(4): 1000-16. doi:S1053-8119(12)00353-9 [pii] 10.1016/j.neuroimage.2012.03.072 [doi]
33. Jespersen S N, Bjarkam C R, Nyengaard J R, et al. Neurite density from magnetic resonance diffusion measurements at ultrahigh field: comparison with light microscopy and electron microscopy. Neuroimage. Jan. 1 2010; 49(1):205-16. doi:S1053-8119(09)00966-5 [pii] 10.1016/j.neuroimage.2009.08.053 [doi]
34. Jespersen S N, Leigland L A, Cornea A, Kroenke C D. Determination of axonal and dendritic orientation distributions within the developing cerebral cortex by diffusion tensor imaging. IEEE Trans Med Imaging. January 2012; 31(1):16-32. doi:10.1109/TMI.2011.2162099 [doi]
35. Genç E, Fraenz C, Schlüter C, et al. Diffusion markers of dendritic density and arborization in gray matter predict differences in intelligence. Nat Commun. May 15 2018; 9(1):1905. doi:10.1038/s41467-018-04268-8 [pii] 4268 [pii] 10.1038/s41467-018-04268-8 [doi]
36. Nazeri A, Schifani C, Anderson JAE, Ameis S H, Voineskos A N. In Vivo Imaging of Gray Matter Microstructure in Major Psychiatric Disorders: Opportunities for Clinical Translation. Biol Psychiatry Cogn Neurosci Neuroimaging. September 2020; 5(9):855-864. doi:S2451-9022(20)30072-0 [pii] 10.1016/j.bpsc.2020.03.003 [doi]
37. Venkatesh A, Stark S M, Stark CEL, Bennett I J. Age- and memory-related differences in hippocampal gray matter integrity are better captured by NODDI compared to single-tensor diffusion imaging. Neurobiol Aging. Aug. 12 2020; 96:12-21. doi:S0197-4580(20)30260-8 [pii] 10.1016/j.neurobiolaging.2020.08.004 [doi]
38. van Berckel B N, Lipsch C, Timp S, et al. Behavioral and neuroendocrine effects of the partial NMDA agonist D-cycloserine in healthy subjects. Neuropsychopharmacology. May 1997; 16(5):317-24. doi:S0893133X96001960 [pii] 10.1016/S0893-133X(96)00196-0 [doi]

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

We claim:

1. A method of treating Major Depressive Disorder (MDD) comprising administering adjunctive D-cycloserine (DCS) or a pharmaceutically acceptable ester of D-cycloserine, or an alkylated D-cycloserine, or a pharmaceutically acceptable precursor of D-cycloserine to augment transcranial magnetic stimulation (TMS) therapy for Major Depressive Disorder (MDD) optionally intermittent or continuous theta-burst stimulation (iTBS or cTBS), high- or low-frequency stimulation and combinations thereof, to improve depressive symptoms and provide higher rates of clinical response and remission.

2. A method of treating Major Depressive Disorder (MDD) in a patient and/or improving and/or alleviating and/or reducing frequency of one or more symptoms of MDD, the method comprising: administering to the patient a low dose of D-cycloserine, a pharmaceutically acceptable ester of D-cycloserine, or an alkylated D-cycloserine, or a pharmaceutically acceptable precursor of D-cycloserine, optionally 1-250 mg per patient; and subjecting the patient to transcranial magnetic stimulation, optionally intermittent or continuous theta-burst stimulation (iTBS or cTBS), high- or low-frequency stimulation or combinations thereof.

3. The method of claim 2, wherein the one or more symptoms of MDD include feelings of sadness, tearfulness, hopelessness, short temper, irritability, loss of interest/lack of pleasure, concentration impairments, indecision, memory loss, flat affect, sleep disorders, tiredness, reduced or increased appetite, weight loss or weight gain, feelings of worthlessness or guilt, thoughts of death and/or suicide.

4. (canceled)

5. The method of claim 2, wherein the low dose is a dose of D-cycloserine sufficient to achieve a plasma concentration of D-cycloserine of 1-30 μg/mL.

6. (canceled)

7. The method of claim 2, wherein the method improves and/or reverses and/or partially reverses cognitive impairments in MDD, including working memory and overall subjective cognitive function.

8. The method of claim 7, wherein the low dose is a dose of D-cycloserine sufficient to achieve a plasma concentration of D-cycloserine of 1-30 μg/mL.

9. The method of claim 2, wherein the method reduces suicide risk and suicidal ideation in MDD.

10. The method of claim 9, wherein the low dose is a dose of D-cycloserine sufficient to achieve a plasma concentration of D-cycloserine of 1-30 μg/mL.

11. The method of claim 2, wherein the method improves anxiety symptoms associated with MDD.

12. The method of claim 11, wherein the low dose is a dose of D-cycloserine sufficient to achieve a plasma concentration of D-cycloserine of 1-30 μg/mL.

13. A method of prolonging the effects of transcranial magnetic stimulation, including intermittent theta-burst stimulation, as measured by neurophysiology including electromyography or encephalography, the method comprising: administering to the patient a low dose of D-cycloserine or a N-methyl-D-aspartate receptor agonist or a partial N-methyl-D-aspartate receptor agonist; and subjecting the patient to transcranial magnetic stimulation, optionally intermittent or continuous theta-burst stimulation (iTBS or cTBS), high- or low-frequency stimulation or combinations thereof.
